(19) United States
(12) Patent Application Publication
(10) Pub. No.: US 2014/0080155 A1
(43) Pub. Date: Mar. 20, 2014

(54) DETECTION OF NGAL IN CHRONIC RENAL DISEASE

(71) Applicants: Jonathan M. Barasch, New York, NY (US); Prasad Devarajan, Cincinnati, OH (US); Thomas L. Nickolas, Brooklyn, NY (US); Kiyoshi Mori, Kyoto (JP)

(72) Inventors: Jonathan M. Barasch, New York, NY (US); Prasad Devarajan, Cincinnati, OH (US); Thomas L. Nickolas, Brooklyn, NY (US); Kiyoshi Mori, Kyoto (JP)

(21) Appl. No.: 14/088,638

(22) Filed: Nov. 25, 2013

Related U.S. Application Data
(63) Continuation of application No. 13/650,270, filed on Oct. 12, 2012, now abandoned.
Application No. 13/650,270 is a continuation of application No. 12/567,860, filed on Sep. 28, 2009, now abandoned.
Application No. 12/567,860 is a continuation of application No. 11/374,285, filed on Oct. 13, 2005, now abandoned.
Application No. 11/374,285 is a continuation-in-part of application No. 11/096,113, filed on Mar. 31, 2005.

(51) Int. Cl. G01N 33/68

(52) U.S. Cl. CPC G01N 33/6893
USPC 435/7.92; 436/501

(57) ABSTRACT

Methods of assessing the ongoing kidney status in a subject afflicted with chronic renal failure (CRF) by detecting the quantity of Neutrophil Gelatinase-Associated Lipocalin (NGAL) in fluid samples over time. NGAL is a small secreted polypeptide that is protease resistant and consequently readily detected in the urine and serum as a result of chronic renal tubule cell injury. Incremental increases in NGAL levels in CRF patients over a prolonged period of time are diagnostic of worsening kidney disease. This increase in NGAL precedes and correlates with other indicators of worsening CRF, such as increased serum creatinine, increased urine protein secretion, and lower glomerular filtration rate (GFR). Proper detection of worsening (or improving, if treatment has been instituted) renal status over time, confirmed by pre- and post-treatment NGAL levels in the patient, can aid in designing and/or maintaining a proper treatment regimen to slow or stop the progression of CRF.

### CROSS REFERENCE TO RELATED APPLICATIONS

This application is a continuation of application Ser. No. 12/567,860, filed on Sep. 28, 2009 (pending), which is a continuation of application Ser. No. 11/374,285, filed on Oct. 13, 2005 (abandoned), which is a continuation-in-part of application Ser. No. 11/096,113, filed Mar. 31, 2005 (pending), the disclosures of which are incorporated herein by reference.

### BACKGROUND OF THE INVENTION

Over the past twenty years it has been learned that earlier identification and treatment of kidney disease can result in preventing kidney disease progression. Thus, a biomarker of kidney damage that is able to indicate the presence of both early damage and identify patients at an increased risk of progressive disease would impact kidney disease diagnosis and treatment. Serum creatinine, the current marker of kidney function, is influenced by muscle mass, gender, race, and medications. These limitations often result in the diagnosis of kidney disease after significant damage has already occurred. Higher degrees of damage at diagnosis limit the efficacy of kidney function preservation therapies and result in higher disease progression rates. Our armamentarium against kidney disease relies upon early intervention and includes interrupting the renin-angiotensin system, and aggressive blood pressure, diabetes, and lipid control.

An early marker of kidney damage would promote earlier intervention in order to arrest the progression to end-stage renal disease (ESRD). In order to be of use to the general clinician, the biomarker must indicate renal damage prior to the current indicators of kidney function, be available non-invasively, and be easily interpretable without the use of complex corrections. Neutrophil Gelatinase-Associated Lipocalin (NGAL) has the potential to be an ideal biomarker in chronic kidney disease (CKD) patients.

The practical impact of an early marker of kidney disease is best demonstrated by reviewing the changing demographics of kidney disease. The worldwide epidemic of CKD will double the incidence of ESRD over the next decade, and have a direct impact on healthcare expenditures. Cost estimates have stated that this increase may be up to $16 billion above the current level of spending. In order to control costs, physicians will need to decrease progression rates of CKD to ESRD. Even small decreases in progression rates can result in large economic gains if patients are prevented from requiring renal replacement therapy (RRT). For example, if a decline in the rate of progression to ESRD was achievable at decreased rates of 10%, 20%, and 30%, then the cumulative direct healthcare savings over 10 years would approximately equal $18.56, $39.02, and $60.61 billion, respectively.

The current markers of kidney disease and kidney disease progression are the serum creatinine and urinary protein concentration, including microalbuminuria. The slope of the decrease in GFR has been demonstrated to predict the timing of ESRD, and the level of proteinuria has been shown in multiple studies to correlate with kidney disease progression rates. These are useful biomarkers of kidney disease and its progression that have withstood the scrutiny of multiple studies. However, their ability to recognize early kidney disease is limited. Serum creatinine concentration is recognized as an unreliable measure of kidney function because it is dependent on age, gender, race, muscle mass, weight, and various medications. Correct interpretation of kidney function based on serum creatinine requires complex formulas that are not routinely employed by practicing providers. Although urinary protein is very sensitive for progressive renal disease, its appearance occurs after renal damage has already occurred. A biomarker of early and/or progressive kidney damage should become positive at the earliest point that kidney damage begins to occur. This “subclinical” kidney damage would occur prior to the rise in serum creatinine or even the development of urinary protein. The primary benefit that identification of subclinical kidney damage would confer is the ability to initiate early interventions to promote kidney function preservation. We have already shown that NGAL levels rise before serum creatinine in acute renal failure models in mice and in humans and can be elevated even when tubular damage is not evident by changes in serum creatinine, such as after subtherapeutic doses of cisplatin.

There is an active search for kidney biomarkers that can predict a patient's risk of progressive chronic kidney disease with the hope that early identification of kidney disease will lead to early treatment, or that the biomarker will identify a treatable entity that can depress rates of kidney disease progression. Some examples of promising kidney biomarkers include asymmetric dimethylarginine (ADMA), liver-type fatty acid-binding protein (L-FABP), cystatin C, C-reactive Protein (CRP), and soluble tumor necrosis factor receptor II (sTNFrii). It is not yet clear how these biomarkers will affect chronic kidney disease treatment, how effective they are at detecting the extent of kidney damage, and how they will come into widespread clinical use. It is also not clear how the appearance of these markers occurs with respect to serum creatinine and proteinuria. In fact, none of these biomarkers are known to be a direct measure of kidney damage.

Cystatin C and L-FABP are produced by cells outside the kidney and rely upon filtration across the glomerulus. ADMA is an endogenous nitric oxide synthase (NOS) inhibitor. Elevated levels have been shown to predict kidney disease progression rates. CRP and sTNFrii are measures of inflammatory activity. Their levels have been shown to correlate with kidney disease progression in inflamed states. CRP appears to correlate with endothelial injury, while sTNFrii has been associated with glomerular injury. Out of these biomarkers, only ADMA, CRP, and sTNFrii might represent guides to therapy. However, there is no published literature on their ability to detect preclinical kidney disease. Other potential biomarkers include kidney extracellular matrix probes.

Previous studies have demonstrated that the degree of tubulointerstitial (TI) alterations at renal biopsy are highly correlated with renal function and prognosis. These alterations result from the deposition of extracellular matrix molecules (ECM) in response to renal injury. The use of extracellular matrix probes and extracellular matrix-related (ECMR) probes to assess renal outcomes has recently been reviewed. Although ECM and ECMR probes are promising in their ability to predict the development of microalbuminuria, and progression of renal disease, they are not easily performed because they require a kidney biopsy.

In contrast, NGAL is produced by the nephron in response to tubular epithelial damage and is a marker of TI injury. It has been well established that in ATN from ischemia or nephrotoxicity that NGAL levels rise, even after mild “subclinical” renal ischemia, in spite of normal serum creatinine levels. From preliminary data we know that NGAL is expressed by the CKD kidney of various etiologies, and that elevated urinary NGAL levels are highly predictive of progressive kidney failure. We therefore are studying NGAL in a longitudinal fashion as a noninvasive early marker of kidney function decline in patients with CKD, and compare it with proven biomarkers of kidney disease progression. In addition, we are conducting a pathological series in order to evaluate the characteristics of NGAL expression in the damaged kidney.

In addition to longitudinally comparing NGAL concentrations to serum creatinine, we have decided to include a longitudinal comparison of NGAL to serum Cystatin C levels. Cystatin C is becoming a very important biomarker of kidney disease. Cystatin C has been extensively reviewed. It is a cysteine protease inhibitor produced by all nucleated cells at a constant rate. It has a small molecular weight and it is freely filtered across the glomerulus and it is almost completely reabsorbed and catabolized, but not secreted, by tubular cells. When direct measurements of GFR, such as inulin or iohexol, are used as the gold standard, Cystatin C concentrations outperform creatinine based estimates of GFR, especially at higher values of GFR. However, Cystatin C is not a direct measure of kidney function and it appears that its levels can be affected by factors other than renal function alone. Its concentration has been shown to vary with age, gender, weight, height, cigarette smoking, higher serum C-reactive protein levels, steroid therapy, and rheumatoid arthritis. The full implication of Cystatin C use for the diagnosis and follow-up of CKD will be unknown until further longitudinal studies of Cystatin C are performed. In contrast, because NGAL is a direct marker of tubular damage, it may provide more accurate diagnostic and follow-up information regarding kidney outcome. The inclusion of longitudinal data on Cystatin C will be a significant contribution to the biomarker

An additional aspect of the research generated from the present invention is to establish a repository of urine and serum from patients with CKD whose phenotypes are well characterized. In the current post-genomic era, it is highly likely that enabling technologies such as microarray analysis and proteomics will continue to identify novel predictive biomarkers for CKD. As part of a proposed data sharing plan, our samples will be available to all investigators for testing other emerging biomarkers for CKD. Establishment of a biological repository will also facilitate the acquisition and appropriate storage of biological samples from other centers in the future. The validation of such markers will enable clinical testing of existing or emerging therapeutic and preventive interventions, thus providing new hope and promise in the ongoing battle against the progression of kidney injury to ESRD.

The ability to slow and arrest the progression of chronic renal disease has been a paradigm shift in nephrology. Multiple studies have demonstrated that tight blood pressure and glycemic control, and the use of agents that block the renin-angiotensin system can decrease the rate of decline in kidney function. Earlier and more aggressive treatment of diabetes, hypertension, and proteinuria has been our most effective method to prevent the development and progression of chronic kidney disease. While the recognition and modification of these risk factors has been invaluable, large clinical studies have noted that the incidence and progression of chronic renal disease is dangerously increasing and can vary substantially among the population at risk for kidney disease. Therefore, further improvement in prevention and treatment recommendations must promote earlier identification of patients at a higher risk of disease progression.

Recent guidelines from the National Kidney Foundation (NKF) and the National institute of Diabetes and Digestive Diseases (NIDDK) have called for the identification of new markers of kidney damage. Identification of new markers of risk stratification may result from both biochemical assays as well as from human genetics. We recently discovered a potential risk marker of kidney disease. It is called Neutrophil Gelatinase-Associated Lipocalin (NGAL).

It has been previously demonstrated that NGAL is markedly expressed by kidney tubules very early after ischemic or nephrotoxic injury in both animal and human models. NGAL is rapidly secreted into the urine, where it can be easily detected and measured, and precedes the appearance of any other known urinary or serum markers of ischemic injury. The protein is resistant to proteases, suggesting that it can be recovered in the urine as a faithful marker of tubule expression of NGAL. Further, NGAL derived from outside of the kidney, for example, filtered from the blood, does not appear in the urine, but rather is quantitatively taken up by the proximal tubule. Because of these characteristics we have previously proposed NGAL as a urinary biomarker predictive of acute renal failure. We showed that NGAL is 100% specific and 99% sensitive for the development of acute tubular necrosis (ATN) after cardiac surgery in pediatric patients. Similar data were obtained in a study of adult patients undergoing cardiac revision.

Presently there are no published data on NGAL, expression in the setting of chronic kidney disease (CKD). However, evidence provided in the present invention indicates that NGAL may be predictive not only of acute renal failure but also of worsening kidney function in the CKD population. Given the expected doubling of CKD incidence and prevalence around the globe, and the cost that end-stage renal disease (ESRD) care represents, it is critical to identify a biomarker that is able to predict which patients are at an elevated risk of renal disease progression, so that early therapeutic interventions can be started, and so that medical regimens can be analyzed in a timely fashion. The present invention provides a better understanding of the biological and clinical implications of NGAL on CKD patients. It is expected that NGAL will have a considerable impact on CKD care.

### SUMMARY OF THE INVENTION

The present invention provides methods of assessing the ongoing kidney status in a mammalian subject afflicted with chronic renal failure (CRF) by detecting the quantity of Neutrophil Gelatinase-Associated Lipocalin (NGAL) in fluid samples over time.

One aspect of the invention provides a method for the detection of worsening chronic renal failure in a mammal, comprising the steps of (1) providing a baseline fluid sample from a mammalian subject; (2) providing at least one subsequent fluid sample from the subject; (3) detecting the quantity of NGAL in each sample; and (4) comparing the quantity of NGAL in the subsequent sample to the quantity of NGAL in the baseline sample, an increased quantity in the subsequent sample indicating that renal tubular cell injury is worsening in the subject.

Another aspect of the invention provides a method of monitoring the effectiveness of a treatment for chronic renal failure in a mammal, comprising the steps of: (1) providing a baseline fluid sample from a mammalian subject experiencing chronic renal failure; (2) providing a treatment for chronic renal failure to the subject; (3) providing at least one post-treatment fluid sample from the subject; and (4) detecting for an increased quantity of NGAL in the post-treatment fluid sample as compared to the quantity of NGAL in the baseline fluid sample.

Another aspect of the invention provides method of identifying the extent of chronic renal failure in a mammal over time, comprising the steps of: (1) providing at least one baseline fluid sample from a mammalian subject at a first time; (2) providing at least one subsequent fluid sample from the subject at a time which is subsequent to the first time; (3) comparing the quantity of NGAL in the subsequent sample to the quantity of NGAL in the baseline sample; and (4) determining the extent of the chronic renal failure in the subject over time based on the time for onset of the increased quantity of NGAL in the subsequent fluid sample, relative to the baseline sample.

Typically the mammalian subject is a human patient, and the fluid samples are urine or serum, but can also be saliva, sputum, bronchial fluid, or plasma. Where more than one subsequent sample is drawn, such that there are a plurality of subsequent samples, they are typically provided intermittently from the subject at predetermined times.

Typically the step of detecting the quantity of NGAL in each sample comprises: contacting each sample with an antibody for NGAL to allow formation of an antibody-NGAL complex, and determining the quantity of the antibody-NGAL complex in each sample, wherein the quantity of antibody-NGAL complex is a function of the quantity of NGAL in each sample. The step of contacting each sample with an antibody fur NGAL to allow formation of an antibody-NGAL complex typically involves the step of contacting the sample with a media having affixed thereto the antibody.

Typically the step of determining the quantity of the antibody-NGAL complex in each sample involves contacting the complex with a second antibody for detecting NGAL. Taken further, this step can include the steps of: separating any unbound material of the sample from the antibody-NGAL complex, contacting the antibody-NGAL complex with a second antibody for NGAL to allow formation of a NGAL-second antibody complex, separating any unbound second antibody from the NGAL-second antibody complex, and determining the quantity of the NGAL-second antibody complex in the sample, wherein the quantity of the NGAL-second antibody complex in the sample is a function of the quantity of the antibody-NGAL complex in the sample. Still further, the step of determining the quantity of the NGAL-second antibody complex in the sample can include methods well-known in the art, including the steps of: adding Horseradish peroxidase (HRP)-conjugated streptavidin to the sample to form a complex with the NGAL-second antibody complex, adding a color-forming peroxide substrate to the sample to react with the HRP-conjugated streptavidin to generate a colored product, and thereafter reading the color intensity of the colored product in an enzyme linked immunosorbent assay (ELISA) reader, wherein the color intensity is a function of the quantity of the NGAL-second antibody complex in the sample.

When a chronic injury is the cause of the chronic renal failure, the chronic injury can be caused by any of the following: chronic infections, chronic inflammation, glomerulonephritides, vascular diseases, interstitial nephritis, drugs, toxins, trauma, renal stones, long standing hypertension, diabetes, congestive heart failure, nephropathy from sickle cell anemia and other blood dyscrasias, nephropathy related to hepatitis, HIV, parvovirus and BK virus, cystic kidney diseases, congenital malformations, obstruction, malignancy, kidney disease of indeterminate causes, lupus nephritis, membranous glomerulonephritis, membranoproliferative glomerulonephritis, focal glomerular sclerosis, minimal change disease, cryoglobulinemia, ANCA-positive vasculitis, ANCA-negative vasculitis, amyloidosis, multiple myeloma, light chain deposition disease, complications of kidney transplant, chronic rejection of a kidney transplant, chronic allograft nephropathy, and the chronic effects of immunosuppressives.

### BRIEF DESCRIPTION OF THE DRAWINGS

FIG. 1 shows mean urinary NGAL levels by etiology of CKD.

FIG. 2 shows the log of NGAL and serum creatinine in patients that progressed to endpoint.

FIG. 3 shows the log of NGAL and serum creatinine in patients that did not progress to endpoint.

FIG. 4 shows the log of NGAL and urine protein to creatinine ratio in patients that progressed to endpoint.

FIG. 5 shows the log of NGAL and urine protein to creatinine ratio in patients that did not progress to endpoint.

FIG. 6 shows a Kaplan-Meier Curve fbr Urine NGAL.

FIG. 7 shows a Kaplan-Meier Curve for Urine Protein.

FIG. 8 shows the association between urinary NGAL and percent interstitial fibrosis in kidney biopsy.

### DETAILED DESCRIPTION OF THE INVENTION

As used herein, the phrases “chronic renal tubular cell injury”, “progressive renal disease”, “chronic renal failure (CRF)”, “chronic renal disease (CRD)”, “chronic kidney disease (CKD)” all shall include any kidney condition or dysfunction that occurs over a period of time, as opposed to a sudden event, to cause a gradual decrease of renal tubular cell function or worsening of renal tubular cell injury. For example, chronic kidney disease includes (but is not limited to) conditions or dysfunctions caused by chronic infections, chronic inflammation, glomerulonephritides, vascular diseases, interstitial nephritis, drugs, toxins, trauma, renal stones, long standing hypertension, diabetes, congestive heart failure, nephropathy from sickle cell anemia and other blood dyscrasias, nephropathy related to hepatitis, HIV, parvovirus and BK virus (a human polyomavirus), cystic kidney diseases, congenital malformations, obstruction, malignancy, kidney disease of indeterminate causes, lupus nephritis, membranous glomerulonephritis, membranoproliferative glomerulonephritis, focal glomerular sclerosis, minimal change disease, cryoglobulinemia, Anti-Neutrophil Cytoplasmic Antibody (ANCA)-positive vasculitis, ANCA-negative vasculitis, amyloidosis, multiple myeloma, light chain deposition disease, complications of kidney transplant, chronic rejection of a kidney transplant, chronic allograft nephropathy, and the chronic effects of immunosuppressives.

As used herein the expression “renal tubular cell injury” shall mean a renal or kidney failure or dysfunction, either sudden (acute) or slowly declining over time (chronic), that can be triggered by a number of disease or disorder processes, including (but not limited to): (1) for acute renal tubular cell injury—ischemic renal injury (IRI) including acute ischemic injury and chronic ischemic injury; acute renal failure; acute nephrotoxic renal injury (NRI) toxicity including sepsis (infection), shock, trauma, kidney stones, kidney infection, drug toxicity, poisons or toxins, or after injection with an iodinated contrast dye (adverse effect); and (2) for chronic renal tubular cell injury the diseases and disorder processes listed in the preceding paragraph. Both acute and chronic forms of renal tubular cell injury can result in a life-threatening metabolic derangement.

NGAL is a small secreted polypeptide that is protease resistant and consequently readily detected in the urine and serum as a result of chronic renal tubule cell injury. Incremental increases in NGAL levels in CRF patients over a prolonged period of time are diagnostic of worsening kidney disease. This increase in NGAL precedes and correlates with other indicators of worsening CRF, such as increased serum creatinine, increased urine protein secretion, and lower glomerular filtration rate (GFR). Proper detection of worsening (or improving, if treatment has been instituted) renal status over time, confirmed by pre-- and post-treatment NGAL levels in the patient, can aid the clinical practitioner in designing and/or maintaining a proper treatment regimen to slow or stop the progression of CRF. For example, in acute tubular necrosis (ATN), where NGAL has been primarily studied, its rise anticipates that of serum creatinine by 24-48 hours. In the present invention, it has been determined that NGAL also rises before the serum creatinine in CKD as well. Further, NGAL is non-invasively obtained as it is excreted into the urine at much higher concentrations than in the blood. Finally, in preliminary studies, urinary NGAL concentration was positively correlated with serum creatinine, indicating a possible association between NGAL levels and the extent of tubular damage. In the present invention, it is determined through rigorous clinical and pathological studies that NGAL can detect both early kidney damage and aid in the detection of progression of chronic kidney damage caused by progressive disease.

NGAL levels are measured in patients undergoing therapeutic regimens which control blood pressure, blood glucose, renal hypertension and diets which limit protein intake, all therapies known to reduce the rate of progression of chronic renal disease. NGAL levels are measured during the course of treatment for active glomerulonephritis or glomerulopathy which are chronic diseases of both the renal tubular and renal interstitial compartments. NGAL levels should typically decline during therapy for lupus nephritis, membranoproliferative glomerulonephritis, membranous glomerulonephritis, focal glomerulosclerosis, minimal change disease, cryoglobulinemia, and nephropathy related to hepatitis, HIV, parvovirus and BK virus. NGAL levels are measured and typically decline during treatment for lead cadmium, urate, chemotherapy related nephrotoxicity. Further, NGAL levels are measured and typically decline during treatment for polycystic and medullary cystic kidney disease, as well as for diabetes and hypertension.

### NGAL Expression in In Vitro Models

We have extensively studied NGAL in humans, mice, and rats with normal renal function and in acute renal disease. We found that NGAL is normally secreted into the circulation by the liver and spleen, and it is filtered by the glomerulus and then recovered by the proximal tubule. Here, where NGAL is degraded in lysosomes (from 23 KDa to 14 KDa), and ligands located in the NGAL calyx are released. The capture of circulating NGAL by the proximal tubule is very effective, as little, if any NGAL is found in the urine of normal humans and mice (in humans: filtered load=(21 ng/ml circulating NGAL)×(GFR), whereas urinary NGAL=22 ng/ml. In the mouse: filtered load=(100 ng/ml circulating NGAL)×(GFR), whereas urinary NGAL=40 ng/ml. Even after massive overload of the protein by systemic injections of NGAL (1 mg), there is little protein recovered in the urine. The uptake into the proximal tubule likely reflects the action of megalin. This was ascertained in a megalin knockout mouse that contains a marked increase in urinary NGAL. Only a small amount of degraded NGAL (14,000 Da) is found in the urine, reflecting processing within the kidney. We calculated a plasma t_{1/2}˜10 min that is likely the result of renal clearance. These data stress the specificity of urinary NGAL as a marker of renally derived NGAL,

### NGAL Expression in Models of Acute Renal Failure

In acute diseases such as sepsis and surgical manipulations, including ischemia of the kidney, circulating NGAL levels rose 10³-10⁴ fold. We found that biopsies of human kidney with acute renal failure showed extensive NGAL immunopositive vesicles. These are presumably endocytic vesicles, and they co-localize with markers of lysosomes. Hence in the normal, as in acute renal failure, it appears that an “extra-renal poor” of NGAL delivers the protein to the proximal tubule where it is captured.

Remarkably, circulating NGAL protects renal function even after a severe model of ischemia. Filtered NGAL induces heme-oxygenasel in the proximal tubule, a critical enzyme that maintains the viability of the tubule in the face of different types of stresses, suggesting a mechanism of protection,

In addition to the “extra renal pool” of NGAL (reflected in proximal tubule capture of NGAL), kidney epithelia also expressed the NGAL protein. In normals, there is trace expression in distal tubules. However within 2-6 hours of cross clamping the renal artery or the ureter of mice, rats, pigs, or the kidneys of patients suffering acute renal failure, the renal tubule itself expresses NGAL. By real-time PCR, we found that NGAL mRNA rises 10³ fold. By in situ hybridization in mouse kidney, we found that ischemia induces massive expression of NGAL RNA in the ascending thick limb of the loop of Henle.

Likewise, urinary obstruction induces massive expression of NGAL mRNA in the collecting ducts. In the urine of mice, pigs and humans we detected a 10³-10⁴ fold increase in NGAL protein. A calculation of the fractional excretion of NGAL in human AIN was often greater than one (FE_{NEGAL}>1), confirming that urinary NGAL reflected local synthesis rather than filtration from the blood. This was also the case in patients with prolonged renal failure who were initiating renal replacement therapy. The amount of urinary NGAL was so prodigious in these patients and its response to changes in renal function so rapid that we have used urinary NGAL as a sensitive and predictive marker of acute renal failure in children and in adults undergoing cardiac procedures.

Data shows that in addition to the “extra-renal pool” of NGAL that is cleared by the proximal tubule, renal epithelia (“intra-renal pool”) expresses massive quantities of NGAL which are secreted into the urine. Urinary NGAL is at specific and sensitive marker of acute epithelial damage and indeed it is a reversible marker. Treatment of ischemic mouse kidney with NGAL not only practically negated the rise in creatinine but it also reduced expression of intra renal NGAL message by 70%.

### NGAL Expression in a Model of CKD

It is notable that in our initial evaluation, urine from patients with chronic renal failure contained much more NGAL than was present in the serum (even when corrected for urine creatinine level), suggesting that NGAL not only reflected acute changes in the tubulointerstitial compartment, but also chronic disease. In addition, it has found that NGAL, is one of the most expressed proteins in the ⅘ nephrectomy model of chronic renal disease in two different animal lines. These preliminary data indicate that on the pathological level NGAL, is a potent marker of CKD.

### NGAL Expression in a Population of CKD Patients

We assessed urinary NGAL levels in 91 outpatients from the general nephrology clinic at CUMC that were referred by outside nephrologists for treatment consultation. These were patients with kidney disease resulting from a spectrum of etiologies. Table 1 shows their baseline characteristics. Mean age was 49.2 years and about half the cohort was female. To determine the correlation coefficient between NGAL and other continuous parameters, we log transformed NGAL, along with the serum creatinine, urine albumin to creatinine ratio (UACR) and the total urinary protein. Log NGAL was found to correlate with log serum creatinine at the baseline visit (r=0.54, p<0.0001), the change in serum creatinine between the baseline and follow-up visit (r=0.49, p=0.002), GFR (r=−0.22, p=0.04), log UACR (r=0.55, p<0.0001), and the log of the total urinaty protein (r=0.61, p=<0,0001). There was no correlation between urinary NGAL and age (SD 17.0), systolic blood pressure (SD 15.8), diastolic blood pressure (SD 11.6), weight (SD 24.1), and serum albumin (SD 4.3).

TABLE 1
Baseline Population Characteristics

| | Value |
|---|---|
| Demographics | |
| Age (years - Mean) | 49.2 |
| Female (%) | 47.8 |
| Race (%) | |
| White | 73.9 |
| Black | 10.2 |
| Hispanic | 4.6 |
| Asian | 8.0 |
| Other | 3.4 |
| Clinical Parameters | |
| Systolic Blood Pressure (mmHg - mean) | 135.4 |
| Diastolic Blood Pressure (mmHg - mean) | 81.6 |
| Weight (kg - mean) | 83.3 |
| Laboratory Parameters | |
| Urine NGAL (mcg/dL - mean) | 94.6 |
| Spot Urine Protein (mg/gm - mean) | 3.2 |
| Urine Albumin/Creatinine Ratio (mg/mg - | 2,338.6 |
| mean) | |
| Serum Creatinine (mg/dL - mean) | 2.6 |
| Serum Albumin (g/dL - mean) | 4.2 |
| Estimated GFR (mL/minute - mean) | 46.4 |

Table 2 lists the etiologies of CKD in this cohort. Out of 91 patients, only 81 had assigned diagnoses. The etiology of CKD consisted of 38% glomerulonephritis, 44% nephrotic syndrome, and 17% other causes. The mean urinary NGAL level for all patients was 94.6 ng/mL. Mean urinary NGAL levels by etiology of CKD were 71.2 ng/mL for the group with glomerulonephritis, 101.7 ng/mL for the group with nephrotic syndrome, and 78.2 ng/mL for the group with other etiologies of kidney disease (See FIG. 1). These levels were not statistically different from each other by ANOVA (F test=0.6890).

TABLE 2
Kidney Diagnoses by Pathological Subgroup

| | Percent |
|---|---|
| Nephritic Syndrome (n = 31) | |
| Anti Cardiolipin Disease | 3.2 |
| C1q Nephropathy | 3.2 |
| Chronic GN* | 6.5 |
| Fibrillary GN | 3.2 |
| Immunocomplex GN | 3.2 |
| IgA Nephropathy | 42.0 |
| Membranoproliferative GN | 6.5 |
| RPGN‡ | 3.2 |
| Lupus Nephritis | 29 |
| Nephrotic Syndrome (n = 36) | |
| Amyloid | 2.8 |
| FSGS [symbol] | 47.2 |
| Minimal Change Disease | 16.7 |
| Membranous Nephropathy | 30.6 |
| Nephrotic Unspecified | 2.8 |
| Other (n = 14) | |
| CKD Unspecified | 28.5 |
| Diabetic Nephropathy | 28.6 |
| Lithium Toxicity | 14.3 |
| Polycystic Kidney Disease | 28.6 |

*GN = glomerulonephritis
‡Rapidly Progressive Glomerulonephritis [symbol]
Focal Segmental Glomerulosclerosis

TABLE 3
Population Characteristics by Progression Status

| | | | | | Non | | |
|---|---|---|---|---|---|---|---|
| | n | Progressors | se | n | Progressors | se | p-value |
| Demographics | | | | | | | |
| Age (years - Mean) | 16 | 54.4 | 3.57 | 64 | 49.4 | 2.15 | 0.3 |
| Female (%) | 10 | 55.6 | | 29 | 45.3 | | 0.6 |
| Race (%) | | | | | | | 0.2 |
| White | 12 | 70.6 | | 48 | 76.2 | | |
| Black | 1 | 5.9 | | 6 | 9.5 | | |
| Hispanic | 0 | 0 | | 4 | 6.4 | | |
| Asian | 4 | 23.5 | | 3 | 4.8 | | |
| Other | 0 | 0 | | 2 | 3.2 | | |
| Clinical Parameters | | | | | | | |
| Systolic Blood Pressure | 16 | 141.3 | 4.45 | 63 | 133.7 | 1.97 | 0.1 |
| (mmHg - mean) | | | | | | | |
| Diastolic Blood Pressure | 16 | 83.3 | 2.35 | 63 | 81.0 | 1.56 | 0.3 |
| (mmHg - mean) | | | | | | | |
| Weight (kg - mean) | 15 | 81.4 | 4.79 | 62 | 83.8 | 3.24 | 1.0 |
| Kidney Disease Diagnosis | | | | | | | 0.6 |
| Nephritic Syndrome (%) | 4 | 26.7 | | 25 | 42.4 | | |
| Nephrotic Syndrome (%) | 8 | 53.3 | | 23 | 39.0 | | |
| Other (%) | 3 | 20.0 | | 11 | 18.6 | | |
| Laboratory Parameters | | | | | | | |
| Urine NGAL (μ/dL - mean) | 18 | 294.6 | 46.02 | 64 | 46.6 | 10.90 | <0.0001 |
| Spot Urine Protein (mg/gm - mean) | 7 | 10.2 | 4.07 | 43 | 2.2 | 0.06 | 0.004 |
| Serum Creatinine (mg/dL - mean) | 18 | 4.8 | 0.56 | 63 | 2.0 | 0.16 | 0.0001 |
| Serum Albumin (g/dL - mean) | 13 | 3.4 | 0.26 | 58 | 4.4 | 0.65 | 0.2 |
| Estimated GFR (mL/minute - mean) | 15 | 29.0 | 10.05 | 62 | 49.3 | 3.86 | 0.001 |

### Urinary NGAL Expression and its Relationship to Kidney Disease Progression Status

Table 3 demonstrates the baseline characteristics of the patients stratified on progression to the primary endpoint of a 25% or more increase in serum creatinine or the development of ESRD by the next follow-up visit. We were able to obtain follow-up information on 82 patients out of the original 91. 18 patients (22.0%) of the cohort reached the primary endpoint. Mean urinary NGAL for patients reaching the endpoint was 294.6 ng/mL, while those who did not reach the endpoint had an NGAL level of 46.6 ng/mL (p<0.0001). The group of patients who progressed to endpoint also had a significantly higher mean proteinuria, and a significantly lower mean GFR.

We then constructed linear regression models to assess the relationship between the urinary NGAL and renal function and proteinuria, stratifying on the outcome. In these models NGAL, serum creatinine, and the AUCR was log transformed to normalize the data's distributional properties. The regression coefficients are listed in Table 4. There was a significant linear relationship between log NGAL and log serum creatinine only for patients who progressed to the endpoint,

TABLE 4
Regression Coefficients for Log NGAL and Kidney Parameters

| | Pro- | | p- | Non-Pro- | | p- |
|---|---|---|---|---|---|---|
| Variable | gressors | se | value | gressors | se | value |
| Log Serum Creatinine | 0.28 | 0.1 | 0.01 | 0.23 | 0.1 | 0.1 |
| Total Proteinuria | −0.07 | 0.02 | 0.03 | 16.4 | 3.3 | <0.0001 |
| Log UACR | 0.32 | 0.23 | 0.2 | 0.49 | 0.1 | <0.0001 |

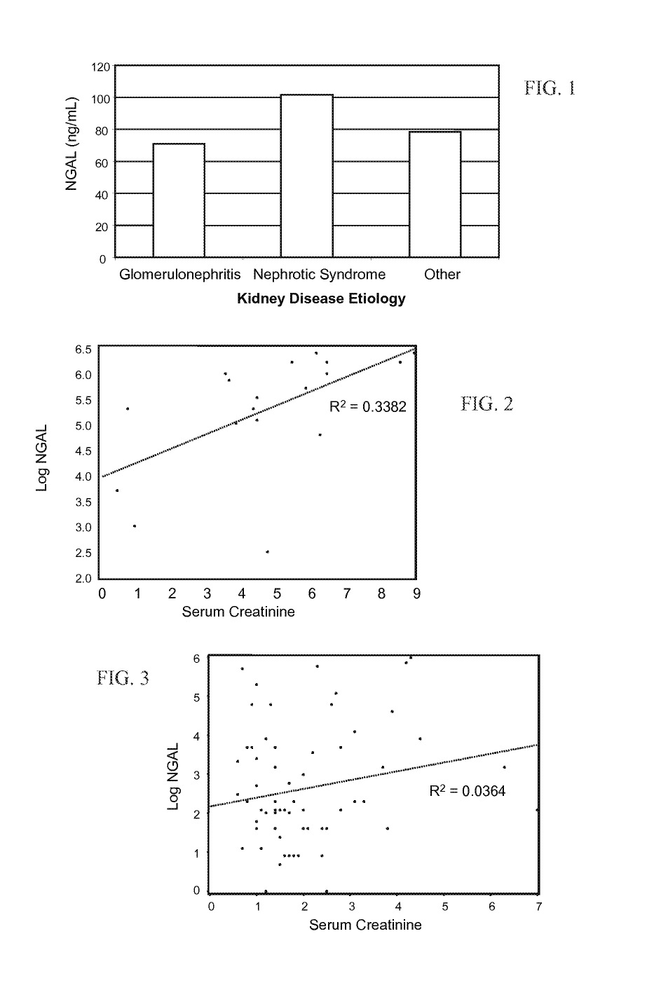

As seen in FIG. 2, in patients who progressed there is a significant linear association in the positive direction between NGAL, and creatinine levels. As seen in FIG. 3, the scatter of data points confirms the non-significant association of NGAL levels and serum creatinine in non-progressors. Stated another way, NGAL levels are very good to have in progressors because they add prognostic information to the serum creatinine

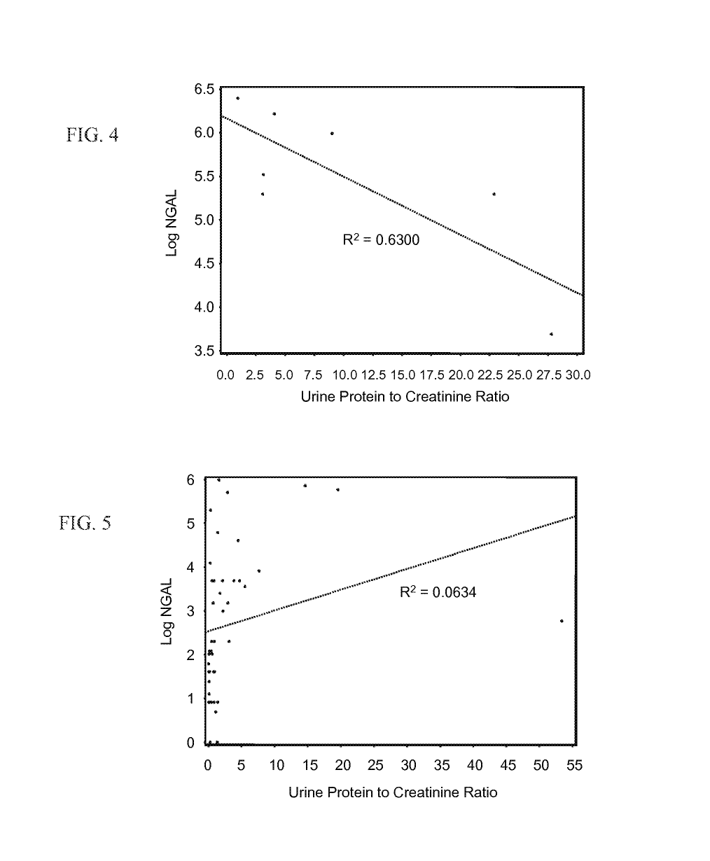

For total proteinuria, regression models demonstrated a significant inverse association between total proteinuria and log NGAL in patients reaching endpoint (FIGS. 4 and 5). There was a linear relationship between log NGAL and log UACR only in those patients that did not progress to endpoint.

### NGAL is Predictive of a Future Decline in Kidney Function

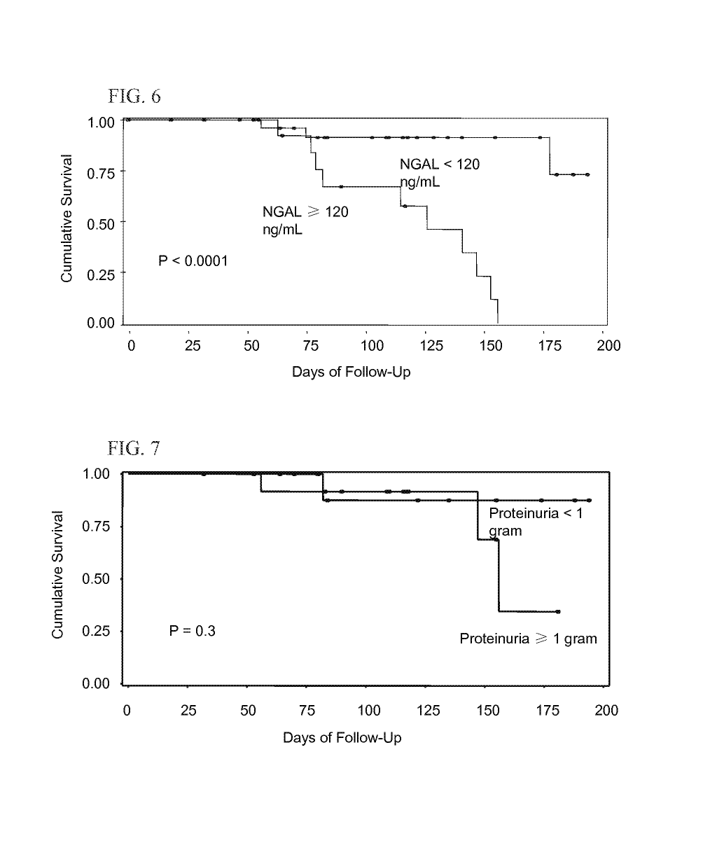

The elevation in urinary NGAL among patients that reached the endpoint led to the hypothesis that NGAL may be an independent predictor of renal function decline. In order to prove this we conducted a sensitivity analysis for both urinary NGAL and urinary protein, an important predictor of progressive renal failure. The primary endpoint was defined as a 25% increase in serum creatinine or the development of ESRD by the time of follow-up. The area under the curve (AUC) for NGAL was 0.908 and that for proteinuria was 0.833. We then defined the cutoff that gave the best sensitivity and specificity for NGAL total proteinuria. At an NGAL concentration 120 ng/mL, the sensitivity was 83.3% and the specificity was 85.9% for predicting the development of poorer renal function at the follow-up visit. For total urinary protein, a cutoff of 1 gram daily demonstrated a sensitivity of 85.7% and a specificity of 81.4%. Using this cutoff, we then proceeded to construct Kaplan-Meier curves for both NGAL and proteinuria (FIGS. 6 and 7). Median survival time for the development of the primary endpoint was 125 days in group with a urinary NGAL≧120 ng/mL (p<K 0.0001). There was no difference in the survival curves for the group with and without proteinuria, as defined by a cutoff of 1 gm daily (p=0.3).

TABLE 5
Hazard Models for the Association of NGAL
Levels with Progressive Kidney Disease

| | Hazard | |
|---|---|---|
| | Ratio | p-value |
| Univariate Proportional Hazard Models | | |
| NGAL (>120 μg/dL) | 12.4 | 0.001 |
| Serum Creatinine (mg/dL) | 1.6 | 0.002 |
| GFR (mL/minute) | 1.0 | 0.2 |
| Proteinuria (>1 gram) | 3.1 | 0.3 |
| Hypertension (SBP ≧ 140 or DBP ≧ 90) | 2.7 | 0.1 |
| Multivariate Proportional Hazard | | |
| Models | | |
| NGAL (>120 μg/dL) | 8.4 | 0.01 |
| Serum Creatinine (mg/dL) | 1.2 | 0.2 |

Further exploration by proportional hazard regression modeling revealed that at a cutoff of 120 ng/mL urinary NGAL was the only independent predictor that remained significantly associated with worsening kidney function at follow-up in a multivariate model (HR 8.4, p<0.01) (See Table 5).

### NGAL and its Relationship to Fibrosis on Kidney Biopsy

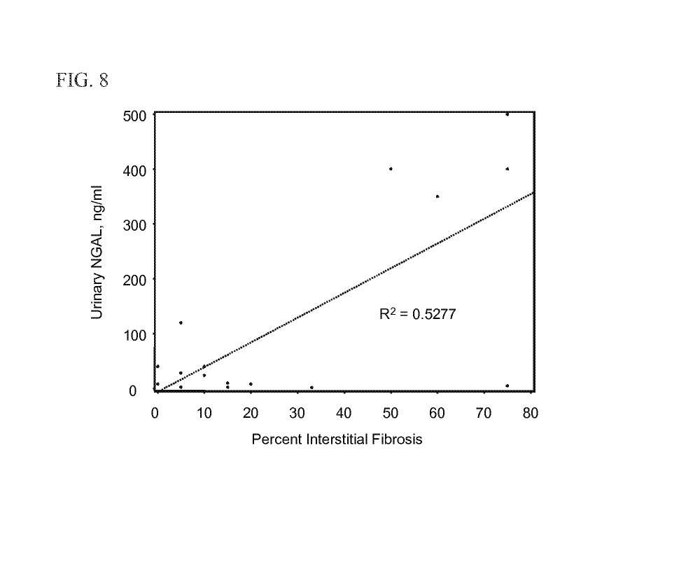

In order to evaluate the relationship between urinary NGAL levels and degree of fibrosis on kidney biopsy, we examined the results of fibrosis scores on 16 kidney biopsy specimens from the cohort of 91 patients. These 16 were chosen because they were read by the renal pathology department at CUMC. These biopsies were obtained up to 2 years prior to the urine NGAL level. Regression analysis indicated that urine NGAL levels Obtained up to 2 years post-renal biopsy were highly correlated with the percent of fibrosis on biopsy (FIG. 8, r²=0.53, p<0.001). We believe this to suggest that NGAL levels are reflective of the chronicity of kidney damage. If this is true, then this is a pathological confirmation of its utility in predicting poor renal outcomes. Collectively, these data indicate an innovative, high-impact development in the discovery and characterization of NGAL as a predictive biomarker for the progression of chronic kidney disease.

While the invention has been described in conjunction with preferred embodiments, one of ordinary skill after reading the foregoing specification will be able to effect various changes, substitutions of equivalents, and alterations to the subject matter set forth herein. Hence, the invention can be practiced in ways other than those specifically described herein, it is therefore intended that the protection herein be limited only by the appended claims and equivalents thereof.

We claim:

1. A method for the detection of worsening chronic renal failure in a mammal, comprising the steps of:
1) providing a baseline fluid sample from a mammalian subject having chronic renal failure;
2) providing at least one subsequent fluid sample from the subject;
3) detecting the quantity of neutrophil gelatinase-associated lipocalm (NGAL) in each sample; and
4) comparing the quantity of NGAL in the subsequent sample to the quantity of NGAL in the baseline sample, an increased quantity of NGAL in the subsequent sample indicating that the chronic renal failure is worsening in the subject.

2. The method according to claim 1, wherein the baseline fluid sample and the at least one subsequent fluid sample are urine samples.

3. The method according to claim 1, wherein the at least one subsequent sample comprises a plurality of subsequent samples obtained intermittently from the subject.

4. The method according to claim 1, wherein the step of detecting the quantity of NGAL in each sample comprises:
A) contacting each sample with an antibody for NGAL to allow formation of an antibody-NGAL complex; and
B) determining the quantity of the antibody-NGAL complex in each sample, wherein the quantity of antibody-NGAL complex is a function of the quantity of NGAL in each sample.

5. The method according to claim 4, wherein the step of determining the quantity of the antibody-NGAL complex in each sample comprises contacting the complex with a second antibody for detecting NGAL.

6. The method according to claim 4, wherein the step of determining the quantity of the antibody-NGAL complex in each sample comprises the steps of:
(i) separating any unbound material of the sample from the antibody-NGAL complex;
contacting the antibody-NGAL complex with a second antibody for NGAL to allow formation of a NGAL-second antibody complex;
(iii) separating any unbound second antibody from the NGAL-second antibody complex; and
(iv) determining the quantity of the NGAL-second antibody complex in the sample, wherein the quantity of the NGAL-second antibody complex in the sample is a function of the quantity of the antibody-NGAL complex in the sample.

7. The method according to claim 6, wherein the step of determining the quantity of the NGAL-second antibody complex in the sample comprises:
a) adding Horseradish peroxidase (HRP)-conjugated streptavidin to the sample to form a complex with the NGAL-second antibody complex;
b) adding a color-forming peroxide substrate to the sample to react with the HRP-conjugated streptavidin to generate a colored product; and
c) thereafter reading the color intensity of the colored product in an enzyme linked immunosorbent assay (ELISA) reader, wherein the color intensity is a function of the quantity of the NGAL-second antibody complex in the sample.

8. The method according to claim 4, wherein the step of contacting each sample with an antibody for NGAL to allow formation of an antibody-NGAL complex comprises the step of contacting the sample with a media having affixed thereto the antibody.

9. The method according to claim 1, wherein the mammalian subject is a human patient.

10. A method of monitoring the effectiveness of a treatment for chronic renal failure in a mammal, comprising the steps of:
1) providing a baseline fluid sample from a mammalian subject experiencing chronic renal failure;
2) providing a treatment for chronic renal failure to the subject;
3) providing at least one post-treatment fluid sample from the subject; and
4) detecting for an increased quantity of NGAL in the post-treatment fluid sample as compared to the quantity of NGAL in the baseline fluid sample.

11. The method according to claim 10, wherein the baseline fluid sample and the at least one post-treatment fluid sample are urine samples.

12. The method according to claim 10, further comprising the step of providing one or more subsequent post-treatment fluid samples, wherein the step of providing treatment is continued until the quantity of NGAL in the subsequent post-treatment fluid samples is either no longer increased or not detected.

13. The method according to claim 10, wherein the step of detecting for an increased quantity of NGAL in the post-treatment fluid sample as compared to the quantity of NGAL in the baseline fluid sample comprises the steps of:
A) contacting each sample with a capture antibody for NGAL to allow formation of a capture antibody-NGAL complex;
B) determining the quantity of the capture antibody-NGAL complex in each sample; and
C) comparing the quantity of the capture antibody-NGAL complex in the at least one post-treatment sample to the quantity of the capture antibody-NGAL complex in the baseline sample, a decreased quantity in the at least one post-treatment sample being an indication that the treatment has been effective.

14. The method according to claim 13, wherein the step of determining the quantity of the capture antibody-NGAL complex in each sample comprises the steps of:
(i) separating any unbound material of the fluid sample from the capture antibody-NGAL complex;
(ii) contacting the capture antibody-NGAL complex with a second antibody for detecting NGAL to allow formation of a NGAL-second antibody complex;
(iii) separating any unbound second antibody from the ⁻NGAL-second antibody complex, and
(iv) determining the quantity of the NGAL-second antibody complex in the sample, wherein the quantity of the NGAL-second antibody complex in the sample is a function of the quantity of the capture antibody-NGAL complex in the sample.

15. The method according to claim 14, wherein the step of determining the quantity of the NGAL-second antibody complex in the sample comprises:
a) adding Horseradish peroxidase (HRP)-conjugated streptavidin to the sample to form a complex with the NGAL-second antibody complex;
b) adding a color-forming peroxide substrate to the sample to react with the HRP-conjugated streptavidin to generate a colored product; and
c) thereafter reading the color intensity of the colored product in an enzyme linked immunosorbent assay (ELISA) reader, wherein the color intensity is a function of the quantity of the NGAL-second antibody complex in the sample.

16. The method according to claim 13, wherein the step of contacting each sample with a capture antibody for NGAL to allow formation of a capture antibody-NGAL complex comprises the step of contacting the sample with a media having affixed thereto the capture antibody,

17. A method of identifying the extent of chronic renal failure in a mammal over time, comprising the steps of:
1) providing at least one baseline fluid sample from a mammalian subject at a first time;
2) providing at least one subsequent fluid sample from the subject at a time which is subsequent to the first time;
3) comparing the quantity of NGAL in the subsequent sample to the quantity of NGAL in the baseline sample; and
4) determining the extent of the chronic renal failure in the subject over time based on the time for onset of the increased quantity of NGAL in the subsequent fluid sample, relative to the baseline sample.

18. The method according to claim 17, wherein the at least one baseline fluid sample and the at least one subsequent fluid sample are urine samples.

19. The method according to claim 17, wherein a surgical procedure has been performed on the subject subsequent to the first time.

20. The method according to claim 17, wherein a chronic injury is the cause of the chronic renal failure, the chronic injury selected from the group consisting of chronic infections, chronic inflammation, glomerulonephritides, vascular diseases, interstitial nephritis, drugs, toxins, trauma, renal stones, long standing hypertension, diabetes, congestive heart failure, nephropathy from sickle cell anemia and other blood dyscrasias, nephropathy related to hepatitis, HIV, parvovirus and BK virus, cystic kidney diseases, congenital malformations, obstruction, malignancy, kidney disease of indeterminate causes, lupus nephritis, membranous glomerulonephritis, membranoproliferative glomerulonephritis, focal glomerular sclerosis, minimal change disease, clyoglobulinemia, ANCA-positive vasculitis, ANCA-negative vasculitis, amyloidosis, multiple myeloma, light chain deposition disease, complications of kidney transplant, chronic rejection of a kidney transplant, chronic allograft nephropathy, and the chronic effects of immunosuppressives.

21. The method according to claim 1, wherein the baseline fluid sample and the at least one subsequent fluid sample are selected from the group consisting of a serum sample and a plasma sample.

22. The method according to claim 10, wherein the baseline fluid sample and the at least one post-treatment fluid sample areselected from the group consisting of a serum sample and a plasma sample.

23. The method according to claim 17, wherein the at least one baseline fluid sample and the at least one subsequent fluid sample are selected from the group consisting of a serum sample and a plasma sample.
